(19) United States
(12) Patent Application Publication
(10) Pub. No.: US 2020/0054718 A1
(43) Pub. Date: Feb. 20, 2020

(54) EGFR/MFN2 Targeted Nanoparticles Particularly Useful For Treating Multidrug Resistant Triple Negative Breast Cancer Through Mitochondrial Fusion Inhibition

(71) Applicant: Lara S Milane, Boston, MA (US)

(72) Inventor: Lara S Milane, Boston, MA (US)

(21) Appl. No.: 16/603,035

(22) PCT Filed: Apr. 4, 2018
(86) PCT No.: PCT/US18/26006

§ 371 Date: Oct. 4, 2019

Related U.S. Application Data
(60) Provisional application No. 62/481,959, filed on Apr. 5, 2017.

(51) Int. Cl. A61K 38/46; A61K 47/69; A61K 47/60; A61K 47/64; A61K 31/337; A61P 35/00

(52) U.S. Cl. CPC A61K 38/46; A61K 47/6909; A61K 47/60; C12Y 306/05; A61K 31/337; A61P 35/00; A61K 47/6425

(57) ABSTRACT

Application for MDR TNBC significantly increasing the efficacy of TNBC treatment and address a global health concern by blocking the ability of mitochondria to fuse together and with other organelles through a nanomedicine therapy. The development of a dual targeted nanomedicine therapy targeting the epidermal growth factor receptor on the surface of TNBC cancers cells and subcellular targeting of mitochondria through mitofusin 2 (MFN2) targeting (mitofusin mediates inter-mitochondrial fusion and fusion of mitochondria with the endoplasmic reticulum). The combination therapy delivers an MFN2-peptidepolymer construct for blocking MFN2 along with a low dose of BAM? (a BAX activator). Transient blocking of MFN2 reduces cellular energy capacity (through decreased mitochondrial fusion), decrease total protein production (by decreased mitochondrial coupling to the endoplasmic reticulum), increases the susceptibility of the cell to paclitaxel or BAM? (increased efficacy of lower dose), with minimal toxicity to normal cells (as IVIFN2 blocking inhibits mitochondrial fusion not mitochondrial function).

### PRIORITY

This application claims the benefit of international application PCT/US2018/026006, filed Apr. 4, 2018, which claim the benefit of U.S. Provisional Patent Application No. 62/481,959, filed Apr. 5, 2017, the contents of all of which are incorporated by reference in their entireties herein.

### TECHNICAL FIELD

There is a real need to advance chemotherapeutic treatment of triple negative breast cancer to reduce the non-specific side effects of chemotherapeutics.

### BACKGROUND ART

Mitochondrial dysfunction is an important hallmark of cellular dysfunction associated with cancer. Cancer cells that fail to go into apoptosis in response to treatment provide a very real barrier to successful combinatorial chemotherapeutic therapy. Cancer driven mitochondrial override mechanisms include but are not limited to: decreased apoptosis, decreased oxidative phosphorylation, and increased aerobic glycolysis. The work of Milane et. al. (2011) demonstrates that transient cellular hypoxia contributes to multi-drug resistance (MDR) in many re-occurring triple negative breast cancer cases (TNBC).

In the past the liver toxicity associated with multi-drug therapies in treating (TNBC) occurrence out weights the positive benefits of using cocktail treatments. In the current invention we disclose a multi-drug therapy that has little or no systemic toxicity that effectively targets the energy systems (mitochondria) in TNBC cancer.

Nanomedicine offers an exceptional opportunity in drug design for new cancer therapies; the opportunity to increase specificity through “active targeting” of molecular residues relevant to a particular phenotype, the opportunity to achieve combination therapy in one formulation, the opportunity to deliver drugs and biologics that cannot be delivered in free¹ solution, and the opportunity to use a lower dose of antineoplastic agents and decrease residual toxicity of treatment. There are over 40 nanomedicine formulations approved by the FDA or equivalent agencies, with almost 10 FDA nanomedicine therapies for cancer treatment and over 15 nanomedicine therapies in clinical trials for cancer treatment [4]. Based on the disclosed advance in this application, a mitochondriotropic nanomedicine therapy for reversing multi-drug resistant (MOR) in TNBC is now a viable and effective treatment approach for managing breast cancer.

### SUMMARY OF INVENTION

In the current invention we disclose the novel application for MDR TNBC; the development of a single and dual targeted nanomedicine therapy that targets the epidermal growth factor receptor on the surface of TNBC cancers cells (this receptor is often overexpressed in TNBC) and subcellular targeting of mitochondria through mitofusin 2 (MFN2) targeting (mitofusin mediates inter-mitochondrial fusion and fusion of mitochondria with the endoplasmic reticulum). The novel therapy will deliver an MFN2-peptide for blocking MFN2 along with a low dose apoptosis activator. Transient blocking of MFN2 reduces cellular energy capacity (through decreased mitochondrial fusion), decrease total protein production (by decreased mitochondrial coupling to the endoplasmic reticulum), increases the susceptibility of the cell to apoptosis activators (increased efficacy of lower dose), with minimal toxicity to normal cells (as MFN2 blocking inhibits mitochondrial fusion not mitochondrial function). Blocking the ability of mitochondria to fuse together and with other organelles through a nanomedicine therapy significantly increase the efficacy of TNBC treatment and address a global health concern.

### BRIEF DESCRIPTION OF DRAWINGS

FIG. 1 is a depiction schema for a treatment schema for cancer treatment employing nanocarriers modified with both EGFR peptide and MFN2 peptide. Treatment schema also includes single targeted MFN2 peptide nanoparticles.

FIG. 2a illustrates MFN2 Fusion (A) of how MFN2 participates in fusion of mitochondrial and endoplasmic reticulum.

FIG. 2b, a MFN2 locking, depicts how mitochondrial fusion may be blocked by MFN2-peptide nanoparticles.

FIG. 3, Nanoparticle Design, depicts a representative nanoparticle design for a targeted MFN2 nanoparticle having PEG modification.

### DESCRIPTION OF EMBODIMENTS

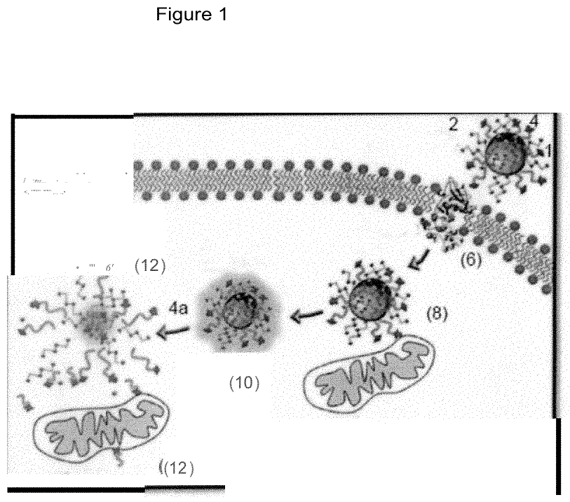

This new drug/treatment scheme is depicted in FIG. 1. The surface of the nanocarriers 1 was modified with an EGFR peptide or PEG 2 and an MFN2 peptide 4. EGFR targeting allowed active targeting of TNBC cells. When the nanocarrier binds to the EGFR receptor (16) it is internalized via a flip-flop mechanism, once inside the cell, the MFN2 residues on the surface of the nanocarrier and in the core of the nanoparticle bind to MFN2 on mitochondria (8) and blocks mitochondrial fusion with each other and with the endoplasmic reticulum. As the nanoparticle degrades, (10) the therapeutics are released (12) and the MFN2-peptide (4a) blocks MFN2 (12) and render a cell susceptible to the proapoptotic agent (14). This treatment scheme improved therapeutic outcomes for MDR TNBC by disabling the bioenergetic network (mitochondrial fusion) and maintaining fission (requirement for apoptosis).

The scheme shown in FIG. 1 can be summarized as:
1. Targeting and binding to MFN2 increases apoptosis as mitochondrial fission is a necessary stage in the apoptotic process.
2. The result of this decreased energy capacity and decreased protein synthesis capacity renders a MDR TNBC cell more susceptible to a low dose of a proapoptotic agent delivered in the nanoparticle formulation (increased efficacy). Dose range 20-200 mg/kg.
3. Dual targeting of EGFR and MFN2 enables specific cell and organelle delivery; through targeting EGFR overexpression in TNBC cells and MFN2 targeting of mitochondria. MFN2 targeting alone is sufficient to disrupt the mitochondrial network in TNBC.
4. The dual targeting system does not cause overt toxicity upon systemic administration as mitochondrial function is not completely inhibited (only mitochondrial fusion will be inhibited); targeting and inhibiting MFN2 with a MFN2-peptide is less toxic than silencing MFN2 through siRNA as blocking MFN2 with the peptide is a transient process.

### A. BACKGROUND AND SIGNIFICANCE

### C.1. Fusion and Fission

Contrary to misconceptions, mitochondria are not static organelles that are merely the “powerhouses” of the cell. Mitochondria are highly plastic organelles that undergo intracellular fission and fusion, out of phase with cell division. Mitochondria are active and mobile, they use the mitochondrial GTP-ase MIRO and its effector MILTON to move bi-directionally along microtubules [5]. Mitochondria certainly function as isolated organelles, but we now know they also function as complex networks to accomplish specific cellular tasks [6]. Mitochondrial copies per cell depend on the function and energy demands of the tissue, with red tissue such as heart having the highest copy number per cell. Mitochondrial morphology is also a tissue variant with hepatocytes having more spherical mitochondria while fibroblast mitochondria are elongated. Perpetual mitochondrial fusion and fission is an important form of cellular quality control, is used to correct for damaged mitochondria, is essential to localizing and migrating mitochondria to specific subcellular regions such as the synapse of a neuron, and is a response to metabolic changes [6, 7]. Not only are mitochondria capable of functioning in networks and in continual contact with each other, but recent biological investigations have revealed that mitochondria are also in direct membrane contact with the endoplasmic reticulum, functioning in mitochondrial fission, in intracellular calcium regulation, and apoptosis [8-11]. Mitochondria have also been reported to have direct membrane association with melanosomes, lysosome related organelles involved in the synthesis and transfer of melanin in pigment cells [9]. Mitochondrial association with the ER and with melanosomes involves similar protein anchors including Mitofusion 2 [9]. Mitochondrial/melanosome contacts have been correlated with melanogenesis [9]. This insight depicts a scenario of mitochondria being recruited to and establishing direct membrane association with organelles undergoing active biogenesis (perpetual contact with ER and transient contact with other organelles as they are involved in biosynthesis). Inhibiting mitochondrial fusion is the targeting therapy in this invention for treating cancer.

### C.2. Mitochondrial Dysfunction in Cancer

Cancer cell mitochondria have long been established as dysfunctional; increased mtDNA mutations, increased ROS production, decreased OXPHOS, and failure to induce apoptosis [12-14]. Due to the central role of mitochondria in programmed cell death and the inherent resistance to apoptosis of cancer cells, cancer is very much a mitochondrial disease. Most types of cancers are resistant to both extrinsic and intrinsic apoptotic signaling [15]. Apoptosome dysregulation has been linked to the carcinogenesis of many different cancers [12]. Mutations in the tumor suppressor gene p53 are the most common mutations in human cancers; p53 functions in apoptosis regulation [16]. Bcl-2, an anti-apoptotic protein, is over-expressed in many tumors conferring resistance of cancer cells to apoptosis [13]. Mitochondria are central to energy and apoptotic dysfunction in cancer.

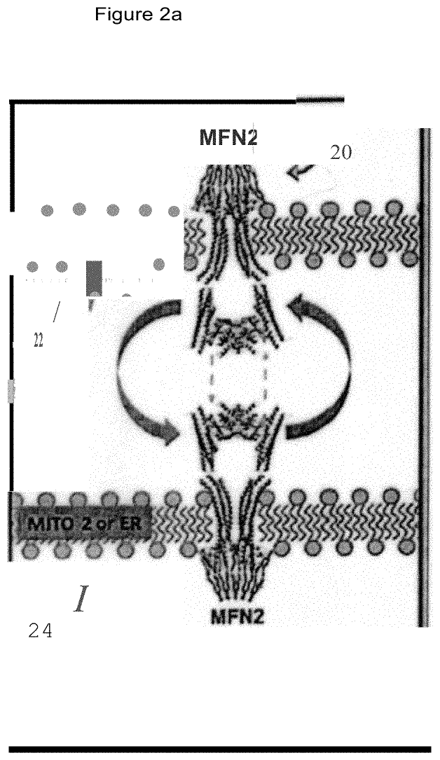

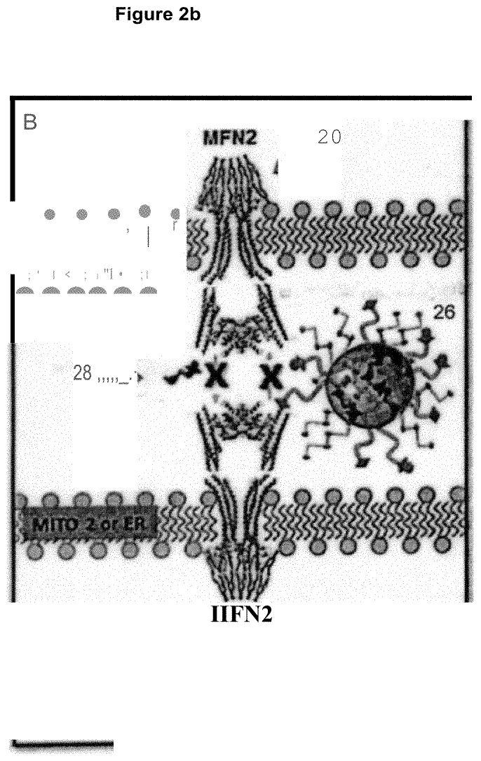

The mechanism of action of MFN2 fusion and therapeutic blocking is demonstrated in FIG. 2. As shown in Panel A mitofusion 2 (20) a protein in humans encoded by the MFN2 gene is embedded in the outer membrane of the mitochondria (22). MFN2 mediates fusion of mitochondria together and the fusion of mitochondria and the endoplasmic reticulum (24). The therapeutic targeted MFN2 nanoparticles (26) of the present invention (Panel B) blocks mitochondrial fusion in conjunction with MFN2-peptide (28). The MFN2 peptide binds to the surface of the nanoparticle and the encapsulated MFN2 peptide functions to block MFN2 mediated mitochondrial fusion.

### Preferred Embodiments

A selection of EGFR positive cells from ATCC's triple negative breast cancer panel is used (MDA-MB-231, BT549, and BT-20) along with SKOV3 ovarian cancer cells and MDR SKOV3 cells (included an established MDR cell line was used as a positive control), and MDA-MB-435 cells (for an EGFR negative control). The MDA-MB-231 cells, BT549, SKOV3, and MDA-MB-435 cells are also part of the NC1-60 Human Cancer Cell Line Screen for developmental therapeutics Hypoxic derivatives of the cell lines were created using a modular incubation chamber were flushed with a 0.5% O2, 5% CO2, nitrogen balanced gas for five minutes and incubated at 37° C. Hypoxic, normoxic, and MOR cells were incubated at 37° C. and maintained in RPM1-1640 media supplemented with 10% fetal bovine serum and 1% penicillin/streptomycin/amphotericin B mixture.

To determine efficacy of MFN2 blocking in the panel of hypoxic, normoxic, and MDR cell lines, a dose response study was conducted with a range of time points and concentrations using MFN2 siRNA (positive control), an MFN2 antibody, an MFN2-peptide, an MFN2-peptide-polymer construct, a combination of the antibody and peptide (competitive binding study between antibody and peptide), and a combination of the antibody and peptide-construct (competitive binding study between antibody and peptide-construct). The MFN2-peptide was used in a second embodiment.

At each time point, the BCA assay is used to measure basal protein concentration of all samples; results are compared to untreated cells (and normalized to cell number) to assess the effect of MFN2 blocking on protein production (possible outcome of decreased mitochondrial binding to the endoplasmic reticulum). To assess ATP concentration, the Mitochondrial ToxGlo Assay (Promega) was used; this assay measures ATP concentration as well as mitochondrial membrane potential (combined provide data for mitotoxicity). Western blots were performed on nucleic and cytoplasmic protein factions to evaluate the MDR phenotype of the cells; proteins to be examined include Epidermal Growth Factor Receptor (EGFR), P-glycoprotein (P-gp; drug efflux pump), Multi-drug Resistance Protein 1 (MRP1; drug efflux pump), Hypoxia Inducible Factor 1a (HIF-1a; transcription factor upregulated in MOR), Hypoxia Inducible Factor 2a (HIF-2a; transcription factor upregulated in MOR), Glucose transporter 1 (Glut-1), Hexokinase II (HXK2; first enzyme of glycolytic pathway), Complex V (CMPLX V; ATP producing unit in oxidative phosphorylation), Cytochrome C (Cyt C; electron transport chain component, apoptosome component), Mitofusin 1 (MFN1; mitochondrial fusion protein); Mitofusin 2 (MFN2; mitochondrial fusion protein also mediates mitochondrial fusion with the endoplasmic reticulum), and Optic Atrophy 1 (OPa-1; inner mitochondrial membrane fusion protein). Cell viability (MTS assay) was conducted.

### Evaluation of Activity (Compound Efficacy) 2

Discovery of the appropriate peptide; develop and optimize a long chain polymer-peptide construct (for surface modification) and a short chain polymer-peptide construct (for encapsulation). The optimum chain to polymer-peptide construct is with a 2500 MW polymer and a 21 amino acid peptide.

An existing anti-mitofusin 2 peptide was used as a guide for initial peptide design and optimization (sigma-aldrich's M6444 corresponding to amino acid residues 557-576 of human MFN2). Using previously established methods of peptide-polymer conjugation, the peptide was linked to a low molecular weight Poly(ethylene glycol) to create a short chain construct and linked to Poly(ethylene glycol)-poly-lactic-glycolic acid conjugate to create a long chain construct [21-23]. The peptide-polymer chemistry will be optimized to promote extended receptor occupancy (MFN2 binding); the constructs will be included for evaluation. NMR will be used to characterize the constructs.

Synthesis and, optimization, and characterization of targeted (MFN2) polymeric and lipid nanoparticles encapsulating MFN2-peptide (short chain) and paclitaxel.

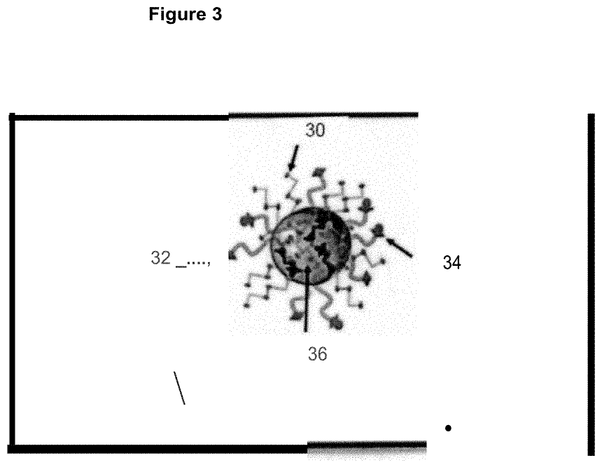

Polymeric nanoparticles were synthesized according to a previously established solvent displacement method [21-23]. Lipid nanoparticles were similarly prepared via lipid film rehydration with the MFN2 peptide in aqueous solution after ten, five minute cycles of liquid nitrogen freezing and heating above the lipid transition temperature (42° C. for 5:3: molar ratio of DOTAP (1,2-dioleoyl-3-trimethylammonium-propane chloride salt) a cationic lipid, cholesterol (stabilizer), and DPPC (1,2-dipalmitoyl-sn-7 glycero-3-phosphocholine) a neutral lipid. The size of the particles was reduced via probe sonication. The previously employed synthesis schema (for combination therapy EGFR-targeted nanoparticles) was modified to include two targeting constructs and adapted to maximize drug encapsulation. Therefore this invention represents a significant advance over the state-of-the-art developed by Milane et. al., (2011). The nanoparticle design is depicted in FIG. 3. PEG modification (30) prevents aggregation of the dual targeted conjugate (32) and MFN2-peptide (34) about a polymer or lipid core (36) in this cure containing MFN2-peptide-fragments and an adjuvant chemotherapeutic agent, combination therapy system. Nanoparticles were characterized for size and zeta potential (using a ZetaPlus Particle Analyzer or similar instrumentation). Surface modification was determined using ESCA analysis and nanoparticles will be imaged by SEM. The optimal dose combination of MFN2-peptide and paclitaxel are determined through a dose response study in the panel of cells. Nanoparticles are loaded with the optimum molar ratio of the agents and loading efficiency was measured by lyophilizing the nanoparticles, rupturing the washed particles, and measuring drug load. Likewise, using lyophilized nanoparticles, drug release kinetics was measured over the time course of 15 minutes to 10 days. The dried nanoparticles are suspended in two different buffers (one at pH 7.4 and one at pH 6.5 to mirror the often acidic microenvironment of a tumor). Samples were removed and quantified (absorbance; plate reader), and buffer was replaced to prevent sink conditions. Burst release phenomena or high drug retention has led to formulation optimization. Active targeting of EGFR and MFN2 was evaluated through competitive (nanoparticles verses antibodies) biding studies, visualized through microscopy.

Assessing the therapeutic efficacy of nanoparticle combination therapy with MFN2-peptides and paclitaxel in MDR, hypoxic, and normoxic cell lines.

Cell viability studies (MTS) were conducted to determine the IC₅₀ values of single agent treatment, combination therapy, in solution, and as nanoparticles. Results are compared to non-targeted nanoparticles (no peptides on surface), unloaded (blank) nanoparticles, media (no treatment), and poly(ethyleneimine) (positive control). The combination index of MFN2-peptide and paclitaxel therapy are determined by comparing combination therapy to solitary treatment. Calculated effective range of doses: 2-200 mg/kg

Safety Studies: Evaluated cellular toxicity and safety of the nanomedicine therapy.

To evaluate the cellular toxicity and safety of the nanomedicine therapy, a panel of non-cancerous cells were evaluated using the MTS assay and the Mitochondrial ToxGlo Assay (to assess mitochondrial toxicity). Toxicity of single agent treatment and combination therapy in solution forms and nanoparticle formulations were evaluated. Toxicity was compared to MFN2 silencing using MFN2 siRNA.

### Data Analysis

Statistical analysis was completed using GraphPad Prism® software and Microsoft Excel.

### INDUSTRIAL APPLICABILITY

Mitochondria are essential for development [18-20]. Mitochondrial fusion and MFN isoforms have yet to be exploited for therapeutic applications. The concept of inhibiting mitochondrial fusion to prevent mitochondria from forming networks to sustain high energy demands, to sensitize to cell death, and prevent fusion with the endoplasmic reticulum to prevent direct energy coupling to protein synthesis is a novel concept for cancer therapy. Inhibiting mitochondrial fusion does improve the efficacy of traditional antineoplastic agents.

### LITERATURE CITED

[1] F. Lara-Medina, V. Perez-Sanchez, D. Saavedra-Perez, M. Blake-Cerda, C. Arce, D. Motola-Kuba, C. Villarreal-Garza, A M. Gonzalez-Angulo, E. Bargall6, J. L. Aguilar, A Mohar, 6. Arrieta, Triple-negative breast cancer in Hispanic patients: high prevalence, poor prognosis, and association with menopausal status, body mass index, and parity, Cancer. 117 (2011) 3658-3669. doi:10.1002/cncr.25961.
[2] M. O. ldowu, M. Kmieciak, C. Dumur, R. S. Burton, M. M. Grimes, C. N. Powers, M. H. Manjili, CD44(+)/CD24(−/low) cancer stem/progenitor cells are more abundant in triple-negative invasive breast carcinoma phenotype and are associated with poor outcome, Hum. Pathol. 43 (2012) 364-373. doi:10.1016/j.humpath.2011.05.005.
[3] A M. Moorman, R. Vink, H. J. Heijmans, J. van der Palen, E. A Kouwenhoven, The prognostic value of tumour-stroma ratio in triple-negative breast cancer, Eur. J. Surg. Oneal. J. Eur. Soc. Surg. Oneal. Br. Assoc. Surg. Oneal. 38 (2012) 307-313. doi:10.1016/j.ejso.2012.01.002.
[4] V. Weissig, T. K. Pettinger, N. Murdock, Nanopharmaceuticals (part 1): products on the market, Int. J. Nanomedicine. 9 (2014) 4357-4373. doi:10.2147/UN.S46900.
[5] E. E. Glater, L. J. Megeath, R. S. Stowers, T. L. Schwarz, Axonal transport of mitochondria requires milton to recruit kinesin heavy chain and is light chain independent, J. Cell Biol. 173 (2006) 545-557. doi:10.1083/jcb.200601067.
[6] E. Braschi, H. M. McBride, Mitochondria and the culture of the Borg: Understanding the integration of mitochondrial function within the reticulum, the cell, and the organism, BioEssays. 32 (2010) 958-966. doi:10.1002/bies.201000073.
[7] R. J. Youle, A M. van der Bliek, Mitochondrial Fission, Fusion, and Stress, Science. 337 (2012) 1062-1065. doi:10.1126/science.1219855.
[8] A S. Rambold, J. Lippincott-Schwartz, SevERing Mitochondria, Science. 334 (2011) 186-187. doi:10.1126/science.1214059.
[9] T. Daniele, M. V. Schiaffino, Organelle biogenesis and interorganellar connections: Better in contact than in isolation, Commun. lntegr. Biol. 7 (2014) e29587. doi:10.4161/cib.29587.
[10] B. Kammann, E. Currie, S. R. Collins, M. Schuldiner, J. Nunnari, J. S. Weissman, P. Walter, An ER-Mitochondria Tethering Complex Revealed by a Synthetic Biology Screen, Science. 325 (2009) 477-481. doi:10.1126/science.1175088.
[11] Mito_ER_Apop_Science-2012.pdf, (n.d.).
[12] J. S. Armstrong, Mitochondria: a target for cancer therapy, Br. J. Pharmacol. 147 (2006) 239-248. doi:10.1038/sj.bjp.0706556.
[13] D. R. Green, G. Kroemer, The pathophysiology of mitochondrial cell death, Science. 305 (2004) 626-629. doi:10.1126/science.1099320.
[14] D. Hanahan, R. A Weinberg, Hallmarks of Cancer: The Next Generation, Cell. 144 (2011) 646-674. doi:10.1016/j.ce11.2011.02.013.
[15] S. Fulda, K.-M. Debatin, Extrinsic versus intrinsic apoptosis pathways in anticancer chemotherapy, Oncogene. 25 (2006) 4798-4811. doi:10.1038/sj.onc.1209608.
[16] K. M. Ryan, A C. Phillips, K. H. Vousden, Regulation and function of the p53 tumor suppressor protein, Curr. Opin. Cell Biol. 13 (2001) 332-337.
[17] M. Suzuki, S. Y. Jeong, M. Karbowski, R. J. Youle, N. Tjandra, The solution structure of human mitochondriafission protein Fis1 reveals a novel TPR-like helix bundle, J. Mol. Biol. 334 (2003) 445-458.
[18] K. N. Papanicolaou, R. Kikuchi, G. A. Ngoh, K. A. Coughlan, I. Dominguez, W. C. Stanley, K. Walsh, Mitofusins 1 and 2 are Essential for Postnatal Metabolic Remodeling in Heart, Circ. Res. 111 (2012) 1012-1026. doi:10.1161/CIRCRESAHA.112.274142.
[19] Mitofusin 2 and Parkin_Damaged Mito Science 2013.pdf, (n.d.).
[20] T. Koshiba, S. A. Detmer, J. T. Kaiser, H. Chen, J. M. Mccaffery, D. C. Chan, Structural basis of mitochondrialtethering by mitofusin complexes, Science. 305 (2004) 858-862. doi:10.1126/science.1099793.
[21] L. Milane, Z. Duan, M. Amiji, Pharmacokinetics and biodistribution of lonidamine/paclitaxel loaded, EGFR-targeted nanoparticles in an orthotopic animal model of multi-drug resistant breast cancer, Nanomedicine Nanotechnol. Biol. Med. 7 (2011) 435-444. doi:10.1016/j.nano.2010.12.009.
[22] L. Milane, Z. Duan, M. Amiji, Development of EGFR-targeted polymer blend nanocarriers for combination paclitaxel/lonidamine delivery to treat multi-drug resistance in human breast and ovarian tumor cells, Mol. Pharm. 8 (2011) 185-203. doi:10.1021/mp1002653.
[23] L. Milane, Z. Duan, M. Amiji, Therapeutic efficacy and safety of paclitaxel/lonidamine loaded EGFR-targeted nanoparticles for the treatment of multi-drug resistant cancer, PloS One. 6 (2011) e24075. doi:10.1371/journal.pone.0024075.

I claim:

1. A method for suppressing cancer cell development comprising:
(a) administering at least one exogenous agent in a therapeutically effective dose which substantially prevents fusion of mitochondria with each other and with the endoplasmic reticulum. (b) administering a known anti-neoplastic agent in a therapeutically effective dose.

2. The method of claim 1 wherein substantial prevention of fusion of the mitochondrial network and mitochondrial fusion to the endoplasmic reticulum entails reducing fusion by more than 95%.

3. The method of claim 1 wherein substantial prevention of fusion of the mitochondrial network and mitochondrial fusion to the endoplasmic reticulum entails reducing fusion by more than 90%.

4. The method of claim 1 wherein substantial prevention of fusion of the mitochondrial network and mitochondrial fusion to the endoplasmic reticulum entails reducing fusion by more than 80%.

5. The method of claim 1 wherein substantial prevention of fusion of the mitochondrial network and mitochondrial fusion to the endoplasmic reticulum entails reducing fusion by more than 70%.

6. The method of claim 1 wherein substantial prevention of fusion of the mitochondrial network and mitochondrial fusion to the endoplasmic reticulum entails reducing fusion by more than 50%.

7. The method of claim 1 wherein at least one exogenous agent comprises an MFN2-peptide and nanoparticle.

8. Polymeric and lipid nanoparticles for use in the treatment of cancer comprising a polymer or aqueous core, a MFN2-peptide, and with and without a EGFR-peptide surface conjugate.

9. The nanoparticles of claim 8 wherein the core contains MFN2-peptide fragments.

10. The nanoparticles of claim 9 wherein the core further comprises an adjuvant chemotherapeutic agent.

11. The nanoparticles of claim 8 further comprising PEG modification about the surface to prevent substantial aggregation of the nanoparticles and avoid immune clearance.

12. The nanoparticles of claim 1 wherein substantial aggregation means no more than 50%.

13. The nanoparticles of claim 1 wherein substantial aggregation means no more than 30%.

14. The is nanoparticles of claim 1 wherein substantial aggregation means no more than 20%.

15. The nanoparticles of claim 1 wherein substantial aggregation means no more than 10%.

16. The nanoparticles of claim 10 wherein the adjuvant chemotherapeutic agent is paclitaxel.
